(19) United States
(12) Patent Application Publication
(10) Pub. No.: US 2006/0189707 A1
(43) Pub. Date: Aug. 24, 2006

(54) Kits and methods for coating a dental filling material to increase wear resistance and durability

(76) Inventors: Norbert Abels, Homburg (DE); Dan E. Fischer, Sandy, UT (US); Claus-H Backes, Saarbrucken (DE)

Correspondence Address: RICK D. NYDEGGER;WORKMAN, NYDEGGER & SEELEY, 1000 Eagle Gate Tower, 60 East South Temple, Salt Lake City, UT, 84111, US

(21) Appl. No.: 11/065,320

(22) Filed: Feb. 24, 2005

(51) Int. Cl. A61K 6/08

(52) U.S. Cl. 523/116

(57) ABSTRACT

Kits and methods for improving the wear and abrasion resistance of filling material involving applying a coating material to at least a portion of a surface of a dental filling material. The dental filling material to be coated is substantially free of organically modified inorganic particles. The coating material includes a polymerizable resin and a plurality of inorganic particles that are organically modified to have polymerizable functional groups thereon. The coating material is cured such that the polymerizable resin and the functional groups on the organically modified inorganic particles co-polymerize or cross-link to form a cured protective coating layer on the dental filling material. The cured coating layer has a maximum thickness of less than about 30 microns.

### BACKGROUND OF THE INVENTION

1. The Field of the Invention

The present invention relates to restorative dental compositions. More particularly the present invention relates to methods of improving the surface properties of a cured dental filling material by providing an abrasive resistant coating on the filling material.

2. The Relevant Technology

Dental fillings have been used for decades to fill and repair cavities and other tooth defects caused by decay or injury. For many years, amalgams and other metal fillings such as gold were used as the filler composition because of their wearability and corrosion resistance.

Recently, new materials have been developed for filling and sealing cavities and root canals. These materials include polymerizable resins such as methacrylates. The polymerizable resins are mixed with fillers and other materials to provide desired properties such as hardness, cure time, and flowability. During recent years, many different polymerizable filling materials have been developed for particular applications.

One problem with many resin based filling materials is that they tend to be softer and less resistant to scratching and wear compared to amalgam fillings. The softness of resin based fillings is typically the consequence of the softness of the polymers used. Hardness and wear resistance in many resins can be increased by adding fillers. However, only so much filler can be added before it adversely affects other properties of the filling material, including bond strength and internal cohesion.

While some resin based filling materials have been developed that have desired hardness and wear resistance properties, these resins often do not have one or more of other desired properties that other compositions may have.

Therefore, what is needed is a method to improve the wear resistance and durability of any resin based filling material such that the filling material can be selected according to beneficial properties. Another need in the art is a method of improving the wear resistance and durability of fillings that have already been placed in a tooth.

### BRIEF SUMMARY OF THE INVENTION

The present invention overcomes the abovementioned problems with resin based filling material by providing kits and methods for applying a protective coating on the surface of a cured filling material. The protective coating increases the wear resistance and durability of the filling material.

In an exemplary embodiment, the present invention includes applying a coating material to at least a portion of a surface of a dental filling material that is itself substantially free of organically modified inorganic particles. The coating material includes a polymerizable resin and a plurality of inorganic particles that are organically modified to have polymerizable functional groups thereon. The coating material is cured such that the polymerizable resin and the functional groups on the organically modified inorganic particles co-polymerize to form a cured protective coating layer on the dental filling material. The protective coating layer preferably has a maximum thickness of less than about 30 microns.

In an exemplary embodiment, the coating material includes inorganic particles that have been organically modified to include functional groups. These functional groups can include amines, vinyl groups, methacrylates, epoxy groups, hydrolyzable and polymerizable silanes, and combinations of these. Suitable polymers include acrylates or methacrylates such as bisphenol-A-glycidyldimethacrylate.

The kits and methods of the present invention provide a protective coating layer that is wear and abrasion resistant. This wear and abrasion resistance is achieved through the cross-linking between the inorganic particles and the polymerizable resin. The cross-linking can connect particles to particles and particles to organic polymer. By creating this web of inorganic and organic molecules, the material can be made resistant to wear and abrasion.

In an exemplary embodiment, the strength and smoothness of the coating layer is due at least in part to the small size of the particles. In one embodiment the particles are less than about 1 micron in diameter, and more preferably less than about 50 nm. The small particle size, high strength, and smoothness of the coating layer also make it possible to apply the coating material in very thin layers. In a preferred embodiment, the cured coating layer has a maximum thickness of less than about 30 microns, more preferably less than about 15 microns, and most preferably less than about 5 microns.

The method of the present invention is particularly advantageous because it can be used with almost any resin based filling material. A dental practitioner can select an appropriate resin based filling material to fill a void or defect with minimal concern of the abrasion and wear resistance of the filling material. The practitioner then applies a layer of the protective coating material over the filling material to provide the desired wear and abrasion resistance.

Exemplary kits include one or more types of dental filling materials and a curable coating material as described herein that is capable of providing increased wear and abrasion resistance.

These and other advantages and features of the present invention will become more fully apparent from the following description and appended claims.

### BRIEF DESCRIPTION OF THE DRAWINGS

To further clarify the above and other advantages and features of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 shows an elevational cut away view of an exemplary tooth with a cavity that that has been cleaned and prepared to receive a filling material;

FIG. 2 shows the tooth of FIG. 1 with the cavity filled with a resin-based filling material and a curing light being used to initiate polymerization of the filling material;

FIG. 3 shows a protective coating material being applied to the surface of the cured filling material of FIG. 2; and

FIG. 4 shows the protective coating material being cured using a curing light.

### DETAILED DESCRIPTION OF THE PREFERRED EMBODIMENTS

I. Introduction

The methods and kits of the present invention are used to perform restorative dental work. First, a tooth is: prepared to receive a filling material using known methods. A filling material is then used to at least partially fill a void or defect. Once the filling material is at least partially cured or hardened, a coating material is applied to the surface of the cured or hardened filling material to improve the wear and abrasion resistance of the filling.

II. Filling Material

A. Polymerizable Resin

In an exemplary embodiment of the present invention, the filling material includes a polymerizable resin material. The polymerizable resin is initially in a liquid or shapeable form. As discussed below, the polymerizable resin is cured to form a polymeric material.

Examples of suitable primary polymerizable resins include a wide range of acrylates, methacrylates, alkylhydroxy methacrylates, alkylamino methacrylates, and derivatives thereof. More specific examples of polymerizable materials include glycidyl dimethacrylate, 2-hydroxy ethyl methacrylate, 3-hydroxy propyl methacrylate, 4-hydroxy butyl methacrylate, triethylene glycol dimethacrylate, and polyethylene glycol dimethacrylate.

In one exemplary embodiment, the polymerizable resin is an oxyphosphorus alkyl methacrylate, such as bis glycerol dimethacrylate phosphate. Examples of other oxyphosphorus alkyl methacrylates within the scope of the invention include bis 2-hydroxy ethyl methacrylate phosphate, phosphate ester of p-hydroxyphenyl methacrylamide, phosphate ester of 3-hydroxy propyl methacrylate, and phosphate ester of 4-hydroxy butyl methacrylate. The oxyphosphorus group increases the adhesiveness and water solubility (i.e. hydrophilicity) of the resulting resin.

One or more additional (or diluent) monomers can be added to achieve the desired properties of initial flowability, curability, and final cured strength and hardness. Diluent monomer suitable for use in the present invention include urethane dimethacrylate, p-hydroxyphenyl methacrylamide, butane diol dimethacrylate, and bisphenol-A-diglycidyl dimethacrylate (“Bis-GMA”).

The primary polymerizable resins are preferably included in a concentration ranging from about 1% to about 90% by weight of the composition, more preferably from about 10% to about 80% by weight, and most preferably from about 20% to about 70% by weight of the composition.

The diluent monomers may be included in amounts of up to about 95% by weight of the composition, preferably in a range from about 10% to about 80%, and more preferably in a range from about 30% to about 70% by weight of the composition.

B. Inorganic Fillers

The filling materials may use any inorganic fillers known in the art in order to improve the strength, hardness, or other desired properties of the filling material. Examples include silica, glass, barium glass, and ceramic particles. In some cases, it may be desirable for inorganic fillers to be radiopaque.

C. Initiators

Initiators are provided in the composition to induce polymerization of the polymerizable material. The initiators or curing agents may include radiant energy polymerization initiators with or without an appropriate organic amine additive or a chemical initiator with an appropriate organic amine additive.

1. Photoinitiators

Examples of photoinitiators within the scope of the invention include camphor quinone, benzoin methyl ether, 2-hydroxy-2-methyl-1-phenyl-1-propanone, diphenyl 2,4,6-trimethylbenzoyl phosphine oxide, benzoin ethyl ether, benzophenone, 9,10-anthraquinone, and derivatives thereof.

Photoinitiators are preferably included in an amount in a range from about 0.05% to about 5% by weight of the composition, more preferably in a range from about 0.1% to about 2% by weight, and most preferably in a range from about 0.2% to about 1% by weight of the composition.

2. Chemical Initiators

Examples of chemical initiators include a wide range of peroxides, other per components, and other free radical generators. A two-part chemical curing system as described more fully below, typically includes a peroxide constituent in one part and an amino compound in another. Exemplary peroxides include benzoyl peroxide, 2-butanone peroxide, lauroyl peroxide and tert-butyl peroxide. Examples of amino compounds include dimethylamino ethyl methacrylate, triethyl amine, 2-dimethylamino ethanol, diethylamino ethyl methacrylate, trihexyl amine, N,N-dimethyl-p-toluidine, N-methylethanolamine, 2,2′(p-tolyimino) diethanol, and derivatives thereof.

In an exemplary embodiment, the chemical initiator is included in the composition in an amount so as to provide sufficient time to allow the dentist or dental practitioner to have sufficient time to place the resinous sealing or filling material into the root canal. In other words, once mixed, the curing time will be sufficiently long so as to allow the dentist to carry out a desired sealing and/or filling procedure.

In most cases, it will be advantageous to include a chemical curing agent (i.e. initiator) in order for the polymerizable resin to cure within a time period of about 5 minutes to about 2 hours, more preferably from about 10 minutes to about one hour and even more preferably from about 10 minutes to about 20 minutes. Such time periods generally provide sufficient time to determine whether or not the sealing material has been properly placed.

Chemical initiators are preferably included in an amount in a range from about 0.01% to about 5% by weight of the composition, more preferably in a range from about 0.05% to about 2% by weight, and most preferably in a range from about 0.1% to about 1% by weight of the composition.

D. Curing

The compositions within the scope of the invention can be chemically curable, photo curable, or dual curable. In the case of chemical and dual curable sealing or filling compositions it is typically necessary to provide a two-part (or multi-part) composition that is mixed by the dentist just prior to use. One part includes constituents of the resin sealing or filling together with one-half of the chemical cure system (e.g., a peroxide compound), while another part includes constituents of the resin sealing with the other half of the chemical cure system (e.g., an amino compound). In the case of a photocurable sealing or filling composition, the polymerizable resin is advantageously stable in the presence of the photoinitiator absent the application of radiant energy.

In the case of chemically curable systems, the final endodontic sealing or filling composition, upon mixing the multiple parts together, preferably cures within a time period of about 15 minutes.

In the case of a photocurable system, including dual cure systems, irradiating the sealing or filling composition with radiant energy, such as from an ultraviolet curing lamp, can effect much more rapid curing than chemical cure (or chemical cure alone). The upper 1-3 mm of sealing or filling material within the root canal or dental preparation is typically photocured in a period of time of about 10 seconds to about one minute.

III. Exemplary Protective Coating Materials

The method of the present invention uses a curable coating material to create a protective coating layer on the surface of a filling material. This coating layer has particular characteristic that give it its hardness and abrasive resistant properties. The coating material is a hybrid of a polymerizable resin and a plurality of organically modified inorganic particles. Generally, the coating material includes at least organically modified inorganic particles, a polymerizable resin, and an initiator for curing the composition.

A. Organically Modified Inorganic Particles

The coating material of the present invention includes a plurality of organically modified inorganic particles. In an exemplary embodiment, the organically modified inorganic particles are made in a two step process. First, metal-alkoxides are subject to hydrolysis and polycondensation in a sol-gel process to form particles of a desired composition. In an exemplary embodiment, the particles have a composition according to the formula MₓO_{y}, where M=Si, Ti, Zr, or Sn. In a preferred embodiment, the particles comprise a ceramic that includes a plurality of Si atoms. In an exemplary embodiment, the inorganic particles have a diameter of less than about 300 microns. More preferably the diameter of the particles is less than about 1 micron.

In a subsequent step the surface of the inorganic particles are functionalized. Functional groups that can be bonded to the surface of the particles include amines, vinyl groups, acrylates, methacrylates, epoxy groups and combinations of these. In a preferred embodiment, the functional groups include a methacrylate such as bisphenol-A-glycidyldimethacrylate.

In an exemplary embodiment, the functionalized inorganic particles include hydrolyzable and polymerizable silanes with the following formula: 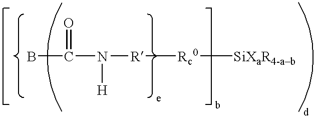
in which the radicals and indices have the following meaning:
B=straight-chain or branched substituted or unsubstituted organic radical having 2 to 50 carbon atoms comprising one or more acrylate and/or methacrylate groups, the C(O)N moiety being bonded to a carbon atom of the radical B;
R=optionally substituted alkyl, alkenyl, aryl, alkylaryl or arylalkyl, each having 1 to 15 carbon atoms, and optionally including oxygen and/or sulfur and/or nitrogen atoms;
R⁰=optionally substituted alkylene, alkenylene, arylene, alkylenearylene or arylenealkylene, each having 1 to 15 carbon atoms, and optionally including oxygen and/or sulfur and/or nitrogen atoms;
R′=optionally substituted alkylene, alkenylene, arylene, alkylenearylene or aryleneakylene, each having 1 to 15 carbon atoms, and optionally including oxygen and/or sulfur and/or nitrogen atoms;
X=hydrogen, halogen, hydroxyl, alkoxy, acyloxy, alkylcarbonyl, alkoxycarbonyl or NR″₂, where R″ is hydrogen, alkyl or aryl;
a=1, 2 or 3;
b=1, 2 or 3, and a+b=2, 3 or 4;
c=0 or 1;
d=1, 2, 3, 4, 5, 6, 7, 8, 9 or 10;
e=1.

Methods of preparing these silanes are disclosed in U.S. Pat. No. 6,794,527 to Wolter et al., issued Sep. 21, 2004, which is incorporated herein by reference. Those skilled in the art will recognize that there are other organically modified inorganic particles that can be used in the polymerizable protective coating material of the present invention.

The foregoing inorganic particles, functional groups, and specific silanes are examples of particle means for increasing abrasion resistance.

B. Polymerizable Resin and Curing System

Another component of the coating material is a polymerizable resin. The polymerizable resin is selected to bond with the functional groups attached to the surface of the inorganic particles. Typically the polymerizable resin can be selected from the same group of polymerizable resins used in the filling material as discussed above. In an exemplary embodiment, the polymerizable resin is an acrylate or methacrylate. In one preferred embodiment, the polymerizable resin is bisphenol-A-glycidyldimethacrylate and/or urethanedimethacrylate (UDMA), which are hydrophobic. Those skilled in the art are familiar with selecting polymerizable resins that give the coating material desired properties.

The coating material also includes a curing system. The curing system is typically selected to work with the polymerizable resin and the functional groups on the inorganic particles. The curing system can be heat curable, light curable, chemically curable, and/or duel curable. The curing system in many cases can be similar to the curing system used to cure the filling material as described above. In a preferred embodiment, the curing system is a light curing system that uses a photoinitiator, which is activated by energy from a UV curing lamp.

A commercially available protective coating material suitable for use in the present invention is ADMIRA, which is sold by Voco, Cuxhaven, Germany. ADMIRA uses an organically modified ceramic known by the trademark ORMOCER, which is made available by Fraunhofer ISC in Wurzburg, Germany. Fraunhofer ISC also makes a coating material suitable for use with the present invention known as ORMOCERE. ADMIRA is particularly advantageous because of it bonds well due to its inclusion of adhesive resins. Another advantage of ADMIRA is that it is transparent and therefore does not significantly alter the aesthetic properties of the filling material.

IV. Applying and Curing the Coating Material

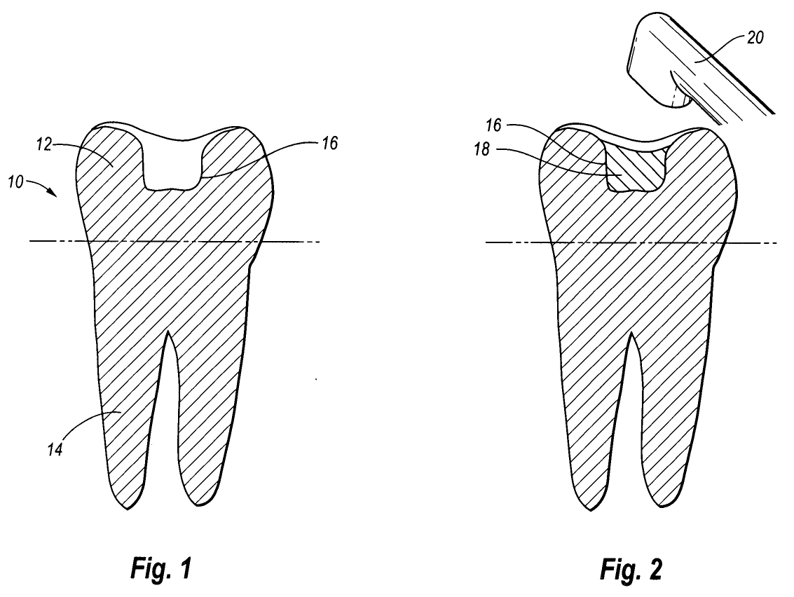

The coating material is applied to at least a portion of a cured filling material to increase the wear and abrasion resistance of the filling material. FIG. 1 shows an exemplary tooth 10 with a crown 12 and a root 14. The crown 12 has a cavity 16 that has been prepared to receive a filling material in a known manner. As shown in FIG. 2, in an exemplary embodiment, a curable filling material 18 is placed in cavity 16 and cured using a curing light 20. The filling material can be any of the filling materials described above that are suitable for use with a light cure system. In an alternative embodiment, other filling materials according the present invention can be used, such as metal amalgams. In addition, cavity 16 can have any shape or size so long as it would require the use of a filling material.

Typically the practitioner selects a filling material that optimizes desired properties. Since a coating layer according to the present invention will be placed on the surface of the filling material, the practitioner can select a filling material that is suitable for a particular patient or complication without worrying about wear and abrasion resistance.

The coating material is applied once the filling material is sufficiently cured or hardened. In one embodiment, the filling material is cured once the surface of the filling material is substantially cured.

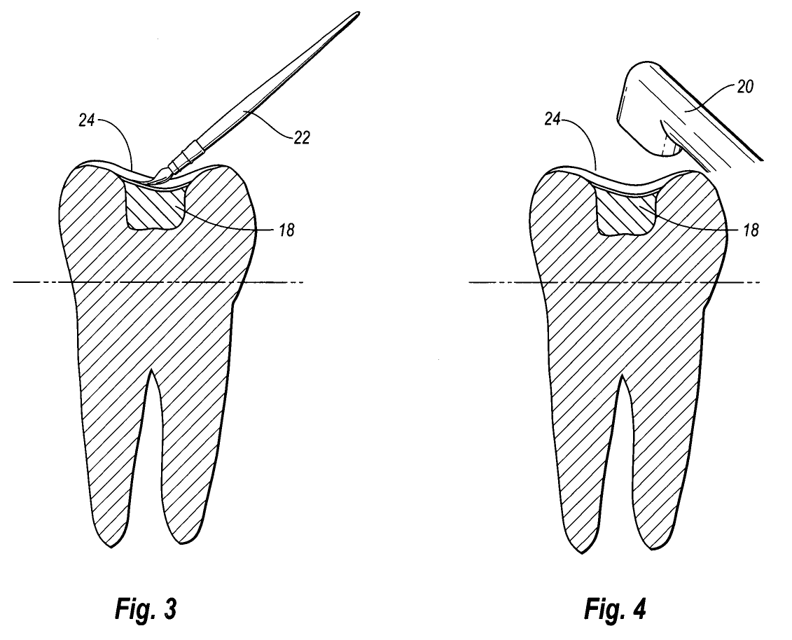

As shown in FIG. 3, in an exemplary embodiment, a coating material is applied to the surface of filling material 18 using a brush 22 to form a coating layer 24. Coating layer 24 is applied to at least a portion of the surface of filling material 18 to improve the wear and abrasion resistance thereof. Other methods of applying the coating material to the surface of the cured filling material 18 can be used, such as spraying or dispensing from a syringe.

As shown in FIG. 4, once the coating material has been applied to at least a portion of the surface of cured filling material 18, the coating layer 24 is cured using a curing light 2. However, other cure mechanisms can be used as desired.

Because of the strength of cured protective coating layer 24, cured coating layer 24 can be made very thin such that it requires very little coating material. In an exemplary embodiment, the coating layer preferably has a thickness of less than about 30 microns, more preferably less than about 15 microns, and most preferably less than about 5 microns.

The filling materials coated according to the methods of the present invention exhibit surprisingly superior properties over other filling materials. For example, a coating layer with a thickness of about 5 microns has a micro harness of about 293 MPa using a Fischer-universal micro-hardness test (DIN 55676) and Berkovich-Diamond indenter. An abrasion test of a 5 micron coating layer evidenced a 1.9% reduction in weight according to the Taber-Abrasion-Test (ASTM D 1044) (using 100 rub-cycles)

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for increasing wear and abrasion resistance of a dental filling material, comprising:
applying a coating material to at least a portion of a surface of a cured dental filling material, the dental filling material being substantially free of organically modified inorganic particles, and wherein the coating material comprises,
a polymerizable resin; and
a plurality of inorganic particles that are organically modified to have polymerizable functional groups thereon; and
curing the coating material such that the polymerizable resin and the functional groups on the organically modified inorganic particles co-polymerize or cross-link to form a cured protective coating layer on the dental filling material, the cured coating layer having a maximum thickness of less than about 30 microns.

2. A method as recited in claim 1, wherein the thickness of the cured coating layer is less than about 15 microns.

3. A method as recited in claim 1, wherein the thickness of the cured coating layer is less than about 5 microns.

4. A method as recited in claim 1, wherein the polymerizable resin comprises a methacrylate or acrylate.

5. A method as recited in claim 4, wherein the methacrylate comprises bisphenol-A-glycidyldimethacrylate.

6. A method as recited in claim 2, wherein the polymerizable resin is light curable.

7. A method as recited in claim 1, wherein the polymerizable resin is heat or chemical curable.

8. A method for increasing wear and abrasion resistance of a dental filling material, comprising:
applying a coating material to at least a portion of a surface of a cured dental filling material, the dental filling material being substantially free of organically modified inorganic particles, and wherein the coating material comprises,
a polymerizable resin; and
a plurality of inorganic particles that are organically modified to have polymerizable functional groups thereon, the organically modified inorganic particles having a size less than about 1 micron in diameter; and
curing the coating material such that the polymerizable resin and the functional groups on the organically modified inorganic particles co-polymerize or cross-link to form a cured coating layer on the dental filling material, the cured coating layer having a maximum thickness of less than about 30 microns and the surface of the cured coating layer results in a maximum abrasion loss of less than about 15% as measured by a Taber Abrasion Test according to ASTM standard D-1044.

9. A method as in claim 8 wherein the cured protective coating layer has an abrasion loss of less than about 10% as measured by a Taber Abrasion Test according to ASTM standard D-1044.

10. A method as in claim 8, wherein the cured protective coating layer has an abrasion loss of less than about 5% as measured by a Taber Abrasion Test according to ASTM standard D-1044.

11. A method as in claim 8, wherein the cured protective coating layer has a micro-hardness greater than about 175 MPa as measured by the Fischer-universal micro-hardness test (DIN 55676).

12. A method as in claim 8, wherein the cured protective coating layer has a micro-hardness greater than about 225 MPa as measured by the Fischer-universal micro-hardness test (DIN 55676).

13. A method as in claim 8, wherein the cured protective coating layer has a micro-hardness greater than about 275 MPa as measured by the Fischer-universal micro-hardness test (DIN 55676).

14. A method as recited in claim 8, wherein the thickness of the cured protective coating layer is less than about 15 microns.

15. A method as recited in claim 8, wherein the thickness of the cured protective coating layer is less than about 5 microns.

16. A kit for increasing the wear resistance of a dental filling material, comprising:
a curable dental filling material suitable for placement onto and bonding to a tooth; and
a protective coating material for placement on a surface of the curable dental filling material after it has been placed onto and bonded to a tooth, the coating material comprising,
a polymerizable resin;
a plurality of inorganic particles that are organically modified to have polymerizable functional groups thereon, the organically modified inorganic particles having a size less than about 1 micron in diameter; and
wherein the polymerizable resin and the polymerizable functional groups co-polymerize or cross-link upon curing of the protective coating material.

17. A kit as recited in claim 16, wherein the polymerizable resin comprises an acrylate or methacrylate.

18. A kit as recited in claim 16, wherein the plurality of inorganic particles comprise a ceramic.

19. An orthodontic bracket as recited in claim 16, wherein the inorganic particles comprise atoms selected from the group consisting of Si, Ti, Zr, Sn, and combinations thereof.

20. An orthodontic bracket as recited in claim 16, wherein the inorganic particles comprise a surface functional group selected from the group consisting of amines, vinyl groups, methacrylates, epoxy groups, hydrolyzable and polymerizable silanes, and combinations thereof.
